(19) United States
(12) Patent Application Publication
(10) Pub. No.: US 2009/0074716 A1
(43) Pub. Date: Mar. 19, 2009

(54) METHOD FOR TREATING INFLUENZA VIRUS INFECTION

(75) Inventors: Ulrich Schubert, Gena (DE); Stephan Ludwig, Muenster (DE); Oliver Planz, Rottenbert (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Virologik GmbH, Erlangen (DE)

(21) Appl. No.: 12/193,237

(22) Filed: Aug. 18, 2008

Related U.S. Application Data
(63) Continuation of application No. PCT/EP2007/051510, filed on Feb. 16, 2007.

(30) Foreign Application Priority Data
Feb. 17, 2006 (DE) 10 2006 008 321.0

(51) Int. Cl. A61K 38/20; A61K 38/02; A61K 31/40; A61K 38/06; A61P 31/16; A61K 31/70; A61K 38/05; A61K 31/69

(52) U.S. Cl. 424/85.2; 514/2; 514/423; 514/18; 514/19; 514/64; 514/23

(57) ABSTRACT

The invention concerns the treatment of orthomyxovirus infections with inhibitors of the ubiquitin protease system, in particular proteasome inhibitors.

### CROSS-REFERENCE TO RELATED APPLICATIONS

The present application is a continuation of PCT/EP2007/051510 filed Feb. 16, 2002 and claims the benefit of DE 10 2006 008 321.0 filed Feb. 17, 2006, the contents of both are incorporated herein by reference.

### BACKGROUND OF THE INVENTION

1. Field of the Invention

The invention concerns the treatment of orthomyxovirus infections with inhibitors of the ubiquitin protease system, in particular proteasome inhibitors.

2. Description of the Related Art

### 1.1. The Influenza Virus Infection in Animals and Humans

Infections with influenza viruses, the influenza pathogen, represent an important threat to health for humans and animals and do not only claim a multitude of fatalities year on year, but are also an immense macroeconomic cost, due to the inability to work caused by illness. Alongside the annually emerging epidemics, influenza nevertheless has an even more menacing dimension, as in the past worldwide outbreaks, so-called pandemics, have come about again and again which have claimed many millions of lives. The emergence of highly pathogenic bird flu viruses of subtype H5N1 over the last few years directly illustrates the danger of a new pandemic in the near future, against which to date no effective vaccines are available.

Influenza viruses belong to the family of orthomyxoviruses and possess a segmented genome of negative-sense orientation, which codes for a minimum of 11 vial proteins. (Lamb and Krug, in Fields, Virology, Philadelphia: Lippincott-Raven Publishers, 1353-1395, 1996). Influenza viruses are arranged into types A, B and C on the basis of molecular and serological characteristics of nucleoproteins (NP) and the matrix proteins (M). Viruses of type A possess the largest pathogenic potential for humans and some animal species (Webster et al., Microbiol Rev, 56, 152-79, 1992).

An influenza A virus particle consists of 9 structural proteins and a lipid coat which comes from the host cell. The viral RNA segments 1 to 3 code for the components of the RNA-dependent RNA polymerase complex (RDRP), PB1, PB2 and PA, connected with the ribonucleoprotein complex catalyse these components transcription and amplification of the viral genome. Haemagglutinin (HA) and neuraminidase (NA) are viral surface glycoproteins which are formed by the vRNA segments 4 and 6. Currently 16 different HA and 9 different NA subtypes are known, and arranged into different categories on the basis of their influenza A viruses.

Viruses with the HA types H1, H2 and H3 and NA types N1 and N2 can be epidemic in humans (Lamb and Krug, in Fields, Virology, Philadelphia: Lippincott-Raven Publishers, 1353-1395, 1996). Segment 5 codes the nucleoprotein (NP), the main components of the ribonucleoprotein complex. Each of the two smallest vRNA segments code for two proteins. The vRNA segment 7 codes the matrix protein M1 and the M2 protein. The M1 protein associates with the inside of the lipid double membrane and clothes the virus cover from the inside out, M2 is a third transmembrane component which functions as a pH-dependent ion channel. The sequence of segment 8 carries the information for the core export protein NS/NEP and the single non-structural protein, NS1. Recently, an eleventh influenza A virus protein was identified (Chen et al., source after the exemplary embodiments) It is about the PB1-F2 protein, which is formed by an open reading frame of the PB1 gene segment shifted around a nucleotide.

PB1-F2 is a mitochondrial protein which is in a position to strengthen the induction of the controlled cellular death, the apoptosis.

The problem of tackling RNA viruses is the viruses' mutability caused by a high error rate of the viral polymerases, which makes both the manufacture of suitable vaccines and the development of antiviral substances very difficult.

It has emerged that the use of antiviral substances which are targeted directly against the functions of the virus leads very quickly to the selection of resistant variants on account of mutation. An example for this is the anti-influenza agent amantadine and its derivations, which are directed against a transmembrane protein of the virus and already lead to the build-up of resistant variants within a small number of passages.

The new therapeutics for influenza infections, which block the influenza viral surface protein neuraminidase and are sold in Germany under the trade names RELANZA and TAMIFLU from Glaxo Wellcome and Roche, have already produced resistant variants in patients (Gubareva et al J Infect Dis 178, 1257-1262, 1998). Resistances to TAMIFLU have even emerged to the H5N1 bird 'flu viruses currently found in humans (Qui et al. Nature 437, 1108, 2005). The hopes which were placed in these therapeutics cannot be fulfilled for this reason.

Due to their mostly small genomes and hence limited coding capacity for functions necessary for replication, all viruses are to a large degree reliant on the functions of their host cells. By the exercise of influence on such cellular functions as are necessary for viral replication, it is possible to negatively affect the virus replication in the infected cell (Ludwig et al., Trends Mol. Med. 9, 46-51, 2003). Here the virus does not have the opportunity to replace the absent cellular function by adaptation. An escape by mutation ahead of pressure of selection is not possible here. This could be shown from the example of the influenza A virus, not only with relatively unspecific inhibitors against cellular kinases and methyl transferases (Scholtissek and Muller, Arch Virol 119, 111-118, 1991), but also with kinase inhibitors which selectively attack a signal pathway required by the virus (Ludwig et al., FEBS Lett 561, 37-43, 2004).

### 1.2. Function of the Ubiquitin/Proteasome System (UPS)

Proteasomes represent the primary proteolytic components in cells and cytosol of all eukaryotic cells. They are multicatalytic enzyme complexes which make up approx. 1% of the total cellular proteins. Proteosomes exercise a vital role in variegated functions of the cellular metabolism. The primary function is proteolysis of miss-folded, non-functional proteins. A further function is possessed by the proteosomal decomposition of cellular or viral proteins for the immune response conveyed by T-cells through the generation of peptide ligands for major histocompatibility class I molecules (for review see Rock and Goldberg, 1999). Proteosome targets are, as a rule, marked by the attachment of oligomeric forms of ubiquitin (Ub) for the decomposition. Ub is a highly conserved, 76 amino-acid-long protein, which is covalently bonded to target proteins. The ubiquitylation itself is reversible, and Ub molecules can be removed again by a multiplicity of Ub hydrolases from the target molecule. The connection between the ubiquitylation of target proteins and the proteasomal proteolysis is generally called a ubiquitin/proteasome system (UPS) (for review see Rock and Goldberg, 1999; Hershko and Ciechanover, 1998).

The 26S proteasome is a 2.5 MDa large multi-enzyme complex, which consists of approx. 31 subunits. The proteolytic activity of the proteasome complex is implemented by a cylindrical, 700 kDa large core structure consisting of four rings lying on top of each other, the 20S proteasome. The 20S proteasome forms an intricate multi-enzyme complex consisting of 14 non-identical proteins, a complex which is arranged in two α- and two β-rings in a αβα-sequence. The substrate specificity of the 20S proteosome comprises three essential activities: Trypsin-, chymotrypsin and postglutamyle-peptide hydrolysing (PGPH) or even casepase-like activities, which are localised in the β-subunits Z, Y and Z. The 20S proteosome degrades in vitro denaturing proteins independently of their polyubiquitylation. On the other hand, in vivo enzymatic activities of the 20S proteasomes are regulated by attachment of the 19S regulatory subunits, which together form the active 26S proteasome particle. The 19S regulatory subunits are involved in the identification of polyubiquitylated proteins as well as in the unfolding of target proteins. The activity of the 26S proteasome is ATP-dependent and degrades almost exclusively only polyubitquitylated proteins (for a review see Hershko and Ciechanover, 1998).

### 1.3. Proteasome Inhibitors

Various drug classes are known as proteasome inhibitors. For one, there are chemically-modified peptide aldehydes like tripeptide aldehyde N-carbobenzoxyl-L-leucinyl-L-leucinyl-L-leucinyl (zLLL; also called MG132) as well as the effective boric acid derivate MG232. Similarly to zLLL, a further class of modified peptides, the peptide vinyl sulphones, was described as proteasome inhibitors (for review see Elliott and Ross, 2001). Naturally-occurring substances are lactacystine (LC) (Fenteany et al., 1995), which is recovered from streptomycetes as well as epoxomycin, which is recovered from actinomycetes (Meng et al., 1999a,b). LC is a highly specific, irreversibly-effective proteasome inhibitor which primarily blocks the chymotrypsin and the trypsin-like activities of the 26S proteasome particle (Fenteany et al., 1995). LC has no peptide basic structure, but rather consists of a γ-lactam ring, a cysteine and a hydroxy-butyl group. LC itself does not inhibit the proteasome. Rather, the N-acetyl cysteine residue is hydrolysed in an aqueous solution. The result is the formation of a clastolactacysteine β-lacton, which is capable of penetrating into the cellular membrane. After accepting the cell, β-lactone-rings and subsequent transesterfication of the threonin-1-Hydroxyl-group of the β-subunit (Fenteany et al., 1995).

With regards to specificity and effectiveness, epoxomycin is the most effective of all known natural proteasome inhibitors to date (Meng et al., 1999; a, b). An additional and very potent class of synthetic proteasome inhibitors are boric acid-peptide derivates, in particular the compound pyranosyl-phenyl-leucinyl-boric acid with the name “PS-341”. PS-341 is very stable under physiological conditions and biologically available after intravenous application (Adams and Stein, 1996; Adams et al., 1999, U.S. Pat. No. 1,448,012TW01).

### 1.4. Clinical Application of Proteasome Inhibitors

The blocking of the proteasome activity as principle cellular protease can lead to alterations in the regulation of the cell cycle, the transcription, the whole cellular proteolysis as well as the MHC-I antigen processing (for review see Ciechanover et al., 2000). As a result, a lasting inhibition of all enzymatic activities of the proteasome cannot be combined with the life of a cell and therefore of the whole organism. Particular, reversibly-acting proteasome inhibitors can, however, inhibit individual proteolytic activities of the 26S proteasome selectively, without thereby influencing other cellular proteases. The first clinical studies with proteasome inhibitors (Adams et al., 1999) make clear the fact that this substance class has an enormous potential as a pharmaceutical with a variegated basis for use (for review see Elliot and Ross, 2001). The meaning of proteasome inhibitors as a new therapeutical principle has taken on increasing notice in the last few years, in particular in the treatment of cancer and inflammatory illnesses (for review see Elliot and Ross, 2001). The proteasome inhibitors developed by the “Millennium Inc.” company (Cambridge, Mass., USA) for antiinflammatory, immunomodulatory and antineoplastic therapies, in particular boric acid derivates of di-peptides and thereby in particular the compound PS-341 (Adams et al., 1999).

The application of proteasome inhibitors with the goal of blocking viral infections has already been described. In particular, it was shown by Schubert et al. (2000 a, b) that proteasome inhibitors block the assembly, release and proteolytic maturation of HIV-1 and HIV-2. This effect is based on a specific blocking of the proteolytic processing of the gag-polyprotein by the HIV protease without proteasome inhibitors influencing the enzymatic activity of the viral protease itself. Further connections with the UP were reported for Budding of the Rous Sarcoma Virus, RSV (Patnaik et al., 2000); Simian Immunodeficiency Virus, SIV (Strack et al., 2000), and Ebola-Virus (Harty et al., 2000). In the latter case (Harty et al., 2000) it was shown that a cellular ubiquitin ligase interacts with ebola matrix protein.

### 1.5. Proteasome Inhibitors in the Patent Literature

Proteasome inhibitors and their medical use are the subject of numerous patents and patent applications. In U.S. Pat. No. 5,780,454 A (Adams et al.), boric acids and ester compounds, their synthesis and use as proteasome inhibitors were described. The inhibition of NF kappa B in a cell is given as the mechanism of proteasome inhibition.

The international patent application WO 98/10779 has as its subject the use of proteasome inhibitors for the treatment of parasite infections.

In the text WO 99/15183, proteasome inhibitors were put to use for the treatment of autoimmune diseases. This is also to do with the role of the UPS in the NFκB-conveyed activation of the HIV-1 LTR promoter, and to do with transcription processes in the cellular nucleus, which are, however, not essential for a replication of HIV. It is not shown that proteasome inhibitors can block the replication of HIV. The NFκB pathway is certainly not suited to that.

Further medical uses are the treatment of fibriotic diseases (US 2005/222043 A), the prevention of a transplant rejection and of a septic shock (EP 0967976 A), the treatment of blood vessel constrictions (WO02/060341 A) or the treatment of an endothelium failure (WO2004/012732 A).

The use of proteasome inhibitors in a cardological indication is also mentioned (DE 10040742 A).

The application of proteasome inhibitors for the treatment of viral infections is the subject of the patent application EP 1430903 A1=US2004/0106539A1. The use of proteasome inhibitors as a means of blocking release, the maturation and the replication of retroviruses is described. Using the example of Human Immunodeficiency Virus (HIV) it is shown that proteasome inhibitors block both processing of the gag proteins and the release of virus particles as well as the infectivity of the released virus particles and with it the virus replication. Areas of use are retroviral therapy and prevention in infections with immunodeficiency-causing lentiviruses in animals and humans, in particular AIDS or HIV-induced dementia, including in combination with other anti-retroviral medicines.

In patent application EP 1326632 A1, means for the treatment, therapy and blocking of hepatitis virus infections and the hepatopathogenesis are diseases are named. The drug used for blocking the release, maturation and replication of hepatitis viruses in pharmaceutical preparations contain as their active ingredients substance classes which have in common the fact that they inhibit the 26S proteasome in cells. This includes above all proteasome inhibitors which influence the activities of the ubiquitin/proteasome pathway, in particular the enzymatic activities of the 26S and the 20S proteasome complex. The use of the invention lies in the antiviral therapy of hepatitis infections, especially in the prevention of the establishment as well as the maintenance of an acute and chronic HBV and HCV infection and liver carcinomas associated with it.

Proteasome inhibitors are also used for the treatment, therapy and blocking of flaviviridae virus infections (WO2003/084551 A1). The drugs used for blocking the release, maturation and replication of flaviviridae in pharmaceutical preparations contain as their active ingredients substance classes which have in common the fact that they inhibit the 26S proteasome in cells.

Proteasome inhibitors are also suggested for the treatment of virus infections, in particular infections with corona viruses causing SARS (severe acute respiratory syndrome).

The importance of the ubiquitin-proteasome pathway for the replication of influenza viruses, or even a use of proteasome inhibitors for the prophylaxis and/or the treatment of infections with influenza viruses has not yet been shown.

In the German patent application DE 103 00 222 A1, the use of active ingredients for the blocking of the IAV replications was described, which exclusively inhibit components of the NF-κB-signal pathway. Alongside a multiplicity of relatively specifically-acting NF-κB inhibitors, proteasome inhibitors were also mentioned as possible active ingredients in this invention description without showing sufficient details of any kind of experimental data for this. On the contrary, it was purely speculated that proteasome inhibitors likewise influence the NF-κB signal transmission pathway. The decisive disadvantage of the invention described in DE 103 00 222 A1 is the fact that investigations regarding the effect of proteasome inhibitors on the NF-κB activation in combination with antiviral effect in influenza viruses have gone negatively so far. In order that it could not be shown that it is possible to produce pharmaceutically effective compositions which contain at least one proteasome inhibitor and/or at least one inhibitor of the UPA, and which are suitable for the treatment of an IAV infection. An anti-viral effect of proteasome inhibitors relating to influenza viruses could not be shown in the invention described in DE 103 00 222 A1. On the contrary, the results contained in the present invention description will show that an effectiveness of proteasome inhibitors on the NF-κB activation require so high a dose of proteasome inhibitors as is not obtainable in vivo by standard applications, and moreover would not be medically justifiable because of the toxicity of the known side-effect of the proteasome inhibitors already used clinically in this degree of concentration. As a result, DE 103 00 222 A1 does not teach that proteasome inhibitors are suitable for the production of antiviral pharmaceutical compositions against influenza viruses.

The international disclosure WO 00/33654 A1 describes the use of an HIV-1 protease inhibitor, Ritonavir, as a proteasome inhibitor. This protease inhibitor has an unspecific effect on the proteasome in the concentration of approx. 10 micrograms, i.e. 10000-times smaller than the effective concentration of a specific proteasome inhibitor. Moreover, this extremely high concentration is not physiologically obtainable. In addition, the logical conclusion emerges that such a permanent blocking of the UPS by Ritonaivr as is administered to people infected with HIV in high-dose anti-retroviral therapy (HAART) must lead to extremely toxic side effect due to the permanent disconnection of the 26S proteasome. To date this has not been described for all patients treated with Ritonavir. Likewise, in the disclosure WO 00/33654 A1 there is a lack of any experimental data for this purely theoretical and also demonstrably false assumption, which could have substantiated such an effect. Of course Ritonavir, as an HIV protease inhibitor, does block the HIV activity, but it does not do that by the unspecific effect on the proteasome, but rather because it blocks the HIV protease in the therapeutically-applicable concentrations exclusively and selectively. Ritonavir blocks the 26S proteasome only in the therapeutically non-obtainable high concentrations, and only Ritonavir can do this, none of the other HIV protease inhibitors known to date. Ultimately, this bizarre effect of Ritonavir was represented on the UPS in vitro. Influenza viruses are also mentioned in WO 00/33654 A1 purely theoretically, however this mention is made exclusively in connection with the improvement of the immune status, in particular concerning the activity of CD4⁺ T-cells. A direct anti-viral effect on influenza viruses is not mentioned. Furthermore, this document does not teach that proteasome inhibitors can be used as antiviral drugs for the production of pharmaceutically effect compositions for the treatment of influenza viruses.

The subject of patent application WO 03/064453 A2 are so-called Trojan inhibitors, which consists of proteasome inhibitors and Trojan peptides. These should also be able to be used in the treatment of influenza viruses. However, in this disclosure too there is a lack of experimental evidence that an antiviral effect against influenza viruses is actually achievable by means of these Trojan inhibitors. Furthermore, it cannot be established from this text that a possible effectiveness of these Trojan inhibitors is to be attributed to the specific inhibitors of the 26S proteasome. It must rather be assumed that a specific effectiveness can only be achieved in that proteasome inhibitor is brought to the target cell by means of the Trojan component, wherein there is also a lack of concrete evidence for this.

The text WO 03/064453 A2 therefore does not teach us to use proteasome inhibitors for the treatment of influenza viruses.

Ubiquitin-ligase inhibitors are the subject of WO 2005/007141 A2. Antiviral components, anti-cancer drug and compounds which can be used for the treatment of neurological disruptions are described therein. Influenza viruses are not mentioned. Moreover, neither in the present text nor in other publications are their any indications that ubiquitin ligases disrupt the replication of influenza viruses.

It can be established, in summary, that in all uses to date of proteasome inhibitors, their effect on influenza viruses and a fortiori their therapeutic uses for the treatment of influenza virus infections are not described. The effect of proteasome inhibitors for the treatment of an influenza virus infection has likewise not yet been discussed. Furthermore, it has not yet been tested whether proteasome inhibitors block the assembly and the release of influenza viruses. Likewise, to date no kind of connection between influenza virus infections and the UPS has been reported. To this extent, the use of inhibitors of both cellular ubiquitin ligases and of ubiquitin hydrolases is equally completely novel.

### SUMMARY OF THE INVENTION

The present invention provides treatments for orthomyxoviruses, e.g., influenza virus with the administration of proteosome inhibitors and/or ubiquitin proteosome pathway (UPS) inhibitors.

### BRIEF DESCRIPTION OF THE DRAWINGS

FIG. 1: Aerosol treatment of Balb/c mice with the proteasome inhibitor Progress of the temperature (A) and the activity (B) of mice, which were treated 3 times a day either with the proteasome inhibitor (red) or with the solvent (black). The graphics show the average value of the measurement of 6 animals. Overall, 288 measurements were noted (every 5 minutes).

FIG. 2: Proteasome inhibitors efficiently block the replication of the influenza virus A549 cells (2×10⁶) (A, B) or MDCK cells (4×10⁶) (C) were pre-incubated for 1 h with the noted concentrations of the particular proteasome inhibitors. After pre-incubation, the cells were infected with the avian influenza virus A/FPV/Bratislava/79 (H7N7) (multiplicity of the infection, MOI=0.01). After 8, 24 or 36 h, the medium remainders were collected and the virus titres determined with the help of plaque assay on MDCK cells.

FIG. 3: Antivirally-acting concentrations of the proteasome inhibitors are not toxic for MDCK cells in the observed time period up to 24 h. (A, B, C) MDCK-cells (2×10⁶) were treated with the noted concentrations of the proteasome inhibitors. As a toxicity test, MDCK cells were treated with the apoptosis-releasing substance Staurosporin (0.3 μM) (black thick lines in the diagram). After 16 or 24 h, the adherent cells as well as cells from the remainder are collected and dyed with 50 μg/ml propidium iodide. The analysis was carried out with the help of throughflow cytometry (BD FACScan). The depiction shows the percentage of living cells in comparison to the untreated test.

FIG. 4: Proteasome inhibitors are not in a position to prevent TNFβ-induced IκBα-degradation in the antivirally-acting concentrations. A549-cells (2×10⁶) (A, B) or HEK293-cells (4×10⁶) (C, D) were pre-incubated for 1 h with the noted concentrations of proteasome inhibitors. After pre-incubation, the cells for stimulated for 15 min with 20 ng/ml recombinant TNFα and lysed. The lysate was separated with the help of the SDS gel electrophoresis and transferred onto a nitrocellulose membrane. IκBα-degradation was detected with the help of an IκBα-specific rabbit serum (Santa Cruz Biotechnologies).

FIG. 5: MG132 blocks influenza virus replication. A549 cells were infected with FPV (MOI=1) in the presence of MG132 (10 μM). 24 h p.i. the remainders were collected and the titres of offspring viruses determined by means of plaque assay.

FIG. 6: MG132 blocks virus-induced induction of TRAIL and FasL A549 with FPV (MOI=1) infects and incubates with MG132 (10M) in the medium.24 p.i. the cells were fixed with 4% paraformaldehyde and the anti-TRAIL and anti-CD95L dyed.

FIG. 7: Antiviral effect of MG132 in infected mice. C57B1/6 mice were FPV infected, (10⁴ pfu, intranasal, were treated with MG132 (n=8) (solid line) by cage inhalation, or received not treatment (n=14) (dotted line). For cage treatment, the animals were treated with aerosol of 2 ml of a 1 mM MG132 (sigma) for 5 days. The treatment was carried out daily and began an hour before the initial infection for 5. For testing the weight of the animals was determined daily. The survival curves of mice infected with FPV and MG132-treated and untreated are shown.

### DETAILED DESCRIPTION OF THE INVENTION

The task underlying the invention is to make available drugs which are suitable for the treatment of infections with influenza viruses, thereby such substances which have an antiviral effect on influenza virus infection in animals and humans. The task is solved by the introduction of inhibitors of the UPS. Both proteasome inhibitors and inhibitors of ubiquitin ligases or ubiquitin hydrolases can be used.

According to the invention drugs with an antiviral effect have been developed which contain as active ingredients both proteasome inhibitors and also inhibitors of ubiquitin ligases or ubiquitin hydrolases in pharmaceutical preparations. The novel drugs according to the invention are suitable for prophylaxis and/or therapy of infections with influenza viruses, in particular of the influenza A virus.

Furthermore, the drugs according to the invention can be used for prophylaxis and/or the treatment, therapy and blocking of an infection with orthomyxo viruses. It is shown that the uses of this drug leads to blocking of the spread of the infection and hence the development of the illness in vivo, in the animal model. These drugs can therefore prevent the establishment of an infection with influenza viruses in animals and humans, or rather heal an infection which has already been established.

The task was solved with the help of pharmaceutical preparations which are suited for blocking the release, maturation and replication of influenza viruses, in particular of IAV.

These preparations are characterised in that they contain at least one proteasome inhibitor as an effective component. Furthermore, these medicines can contain other components of the UPS. This concerns ubiquitin ligases and/or ubitquitin hydrolases, i.e. enzymes, which regulated the ubiquitylation of proteins. This task is therefore solved by a combination of proteasome inhibitors on the one hand and by ubiquitin ligases and/or ubiquitin hydrolases on the other hand. In a preferred embodiment of the invention pure proteasome inhibitors are likewise introduced which are distinguished by a high membrane permeability as well as a high specificity for the 26S proteasome of the host cell.

According to an advantageous embodiment of the invention, antiviral effects can be especially enacted in IAV-infected cells. These concern firstly the induction of the apoptosis into the influenza virus-infected cells, and with it the preferred dying off of infected cells in the organism. At the same time, by inhibition of the assembly and maturation of influenza viruses, the release and the production of infectious virus particles is disrupted. In the total of this effect, a therapeutic effect by blockage of the virus replication and the removal of virus-producing cells in the organism can be brought about.

In a further embodiment of the invention, classic proteasome inhibitors can be put to use to combat infections with influenza viruses. For this, inhibitors should above all be used which interreact exclusively with the catalytically active hydroxyl-threonin group of the bet subunit of the 26S proteasome, and therefore only specifically block the proteasome. A further substantial constituent and surprising effect of this development is the observation that the blockage of the UPS preferably induces the dying off (the apoptosis) of influenza virus-infected cells.

The tasks of the invention were solved by the introduction of at least one proteasome inhibitor and/or at least one inhibitor of ubiquitin ligases or ubiquitin hydrolases. According to the invention, drugs for the treatment of virus infections have been developed which in pharmaceutical preparations contain inhibitors of the UPS as an active ingredient, so for the blocking of influenza viruses. According to a preferred embodiment of the invention, substances were applied as proteasome inhibitors which block, regulated or otherwise influence the activities of the UPS.

It is also possible that substances be introduced as proteasome inhibitors which specially influence the enzymatic activities of the complete 26S proteasome complex and the free 20S catalytically-active proteasome structure not assembled with regulatory subunits. These inhibitors can block either one or several or all three primary proteolytic activities of the proteasome (trypsin, chymotrypsin, and the postglutamyl-peptide hydrolysing activities) within the 26S or even the 20S proteasome complex.

A variant of the invention consists in introducing as proteasome inhibitors substances which are taken up by high eukaryote cells and come into interaction after cell acceptance with the catalytic beta subunit of the 26S proteasome, and thereby block all or individual ones of the proteolytic activities of the proteasome complexes irreversibly or reversibly.

In a further variant of the invention, substances are introduced as specific proteasome inhibitors which, after cell acceptance, selectively block individual enzymatic activities of the 26S proteasome and moreover also selectively block particular assembly forms of the proteasome, like for example the immunoproteasome. The immunoproteasome is formed by reassembly as a particular form of the 26S proteasome in particular after stimulation by interferon treatment. The immunoproteasome can also form as a reaction to an IAV infection. To this extent, the specific inhibition of the immunoproteasome is a particular embodiment of the antiviral effect of proteasome inhibitors in IAV infections. According to the invention, because of this such substances are also introduced which selectively inhibit the immunoproteasome.

As another form of the invention, drugs come into use which block the activities of the ubiquitin-conjugating and/or the ubiquitin-hydrolysing enzymes. To this also belong cellular factors which interact with ubiquitin both as monoubiquitin and as polyubiquitin. Polyubiquitinylation generally counts as an identification signal for the proteolysis by the 26S proteasome, and the influence of the ubiquitinylation pathway can likewise regulate the activity of the proteasome.

According to the invention, substances are also introduced as proteasome inhibitors which are administered in various forms in vivo orally in capsule form, with or without cell-specificity-carrying alterations, intravenally, intramuscularly, subcutaneously, by inhalation in aerosol form, or otherwise, have a low cytotoxicity and/or high selectivity for particular cells and organs due to the use of a particular application and dose regime, have no or only insignificant side effects, and have a relatively high metabolic half-life and a relatively small clearance rate in the organism.

The decisive difference of the present invention as compared to the solution described in is that it could be shown according to the invention that the specific effect of proteasome inhibitors on the IAV replication does not contain the NF-κB-signal transfer pathway, but rather concerns a completely different cellular pathway, that is the ubiquitin proteasome pathway (UPS) on the release of infectious Offspring viruses in an IAV infection. The antiviral effect of proteasome inhibitors on an IAV infection could also be demonstrated for the first time experimentally and in vivo. As a result, it could be disproved in the present invention that proteasome inhibitors likewise influence the NF-κB-signal transfer pathway. The results presented in the Examples clearly show that in an antiviral concentration of proteasome inhibitors effective in vivo the NF-κB-pathway is quite clearly not affected.

Proteasome inhibitors that can be used in the context of the invention include, e.g., chemically-modified peptide aldehydes like tripeptide aldehyde N-carbobenzoxyl-L-leucinyl-L-leucinyl-L-leucinyl (zLLL; also called MG132) as well as the effective boric acid derivate MG232. Similarly to zLLL, a further class of modified peptides, the peptide vinyl sulphones, was described as proteasome inhibitors (for review see Elliott and Ross, 2001). Naturally-occurring substances are lactacystine (LC) (Fenteany et al., 1995), which is recovered from streptomycetes as well as epoxomycin, which is recovered from actinomycetes (Meng et al., 1999a,b). LC is a highly specific, irreversibly-effective proteasome inhibitor which primarily blocks the chymotrypsin and the trypsin-like activities of the 26S proteasome particle (Fenteany et al., 1995). LC has no peptide basic structure, but rather consists of a γ-lactam ring, a cysteine and a hydroxy-butyl group. LC itself does not inhibit the proteasome. Rather, the N-acetyl cysteine residue is hydrolysed in an aqueous solution. The result is the formation of a clastolactacysteine β-lacton, which is capable of penetrating into the cellular membrane. After accepting the cell, β-lactone-rings and subsequent transesterfication of the threonin-1-Hydroxyl-group of the β-subunit (Fenteany et al., 1995).

Other suitable proteosome inhibitors include, epoxomycin (Meng et al., 1999; a, b), boric acid-peptide derivates, in particular the compound pyranosyl-phenyl-leucinyl-boric acid with the name “PS-341” (Adams and Stein, 1996; Adams et al., 1999, U.S. Pat. No. 1,448,012TW01), boric acid derivates of di-peptides and thereby in particular the compound PS-341 (Adams et al., 1999), proteasome inhibitors described in U.S. Pat. No. 5,780,454, the relevant disclosure relating to those proteasome inhibitors is incorporated herein by reference, boric acids and ester compounds, their synthesis and use as proteasome inhibitors were described, proteosome inhibitors described in WO 98/10779, WO 99/15183, US 2005/222043, WO02/060341, WO2004/012732 A, DE 10040742 A, US2004/0106539A1, EP 1326632 A1, WO2003/084551 A1, DE 103 00 222 A1, WO00/33654 A1, WO 00/33654 A1, and WO 03/064453 A2, the relevant disclosures relating to those proteasome inhibitors are incorporated herein by reference.

Ubiquitin-ligase inhibitors are described in WO 2005/007141 A2, the relevant disclosures relating to those inhibitors are incorporated herein by reference.

Furthermore, substances are introduced as proteasome inhibitors which, in their natural form, are isolated from micro-organisms or other natural sources, emerge from natural substances by chemical modifications or are manufactured totally synthetically, or a synthesised by genetic therapeutic methods in vivo, or are manufactured by genetic engineering methods in vitro or in micro-organisms. These include

a) naturally-occurring proteasome inhibitors:
Epoxomicin (Epoxomycin) and Eponemycin,
Aclacinomycin A (also called Aclarubicin),
Lactacystine and its chemically-modified variants, in particular the cell membrane-penetrating variant “Clastolactacystein beta-Lacton”,
   b) synthetically manufactured:
modified peptide aldehydes like e.g. N-carbobenzoxy-L-leucinyl-L-leucinyl-L-leucinal (also called MG132 or zLLL), its boric acid derivate MG232; N-carbobenzoxy-Leu-Leu-Nva-H (called MG115); N-Acetyl-L-Leuzinyl-L-Leuzinyl-L-Norleuzinal (called LLnL); N-carbobenzoxy-Ile-Glu(OBut)-Ala-Leu-H (auch bezeichnet als PSI);
Peptides carrying C-terminal Epoxyketone (also called Epoxomicin/Epoxomycin or Eponemycin), Vinyl-sulphone (for example Carbobenzoxy-L-Leucinyl-L-Leucinyl-L-Leucin-vinyl-sulphone or 4-Hydroxy-5-iodo-3-nitrophenylactetyl-L-Leucinyl-L-Leucinyl-L-Leucin-vinyl-sulphone, also called NLVS), Glyoxal- or boric acid residues (for example Pyrazyl-CONH(CHPhe)CONH(CHisobutyl)B(OH)₂), also called “PS-431” or Benzoyl(Bz)-Phe-boroLeu, Phenacetyl-Leu-Leu-boroLeu, Cbz-Phe-boroLeu); Pinacol-Ester—for example Benzyloxycarbonyl(Cbz)-Leu-Leu-boroLeu-Pinacol-Ester;
and
introduced as particularly suitable compounds were peptides and peptide derivates carrying C-terminal Epoxyketon-structures; this includes for example Epoxomicin (molecular formula: C₂₈H₈₆N₄O₇) and Eponemycin (molecular formula: C₂₀H₃₆N₂O₅);
particular dipeptidyl boric acid derivates, in particular the compound PS-296 (8-Quinolyl-sulfonyl-CONH—(CH-Naphthyl)-CONH(—CH-isobutyl)-B(OH)₂); the compound PS-303 (NH₂(CH-Naphtyl)-CONH—(CH-isobutyl)-B(OH)₂); the compound PS-321 (Morpholin-CONH—(CH-Naphthyl)-CONH—(CH-Phenylalanin)-B(OH)₂); the compound PS-334 (CH3-NH—(CH-Naphthyl-CONH—(CH-Isobutyl)-B(OH)₂); the compound PS-325 (2-Quinol-CONH—(CH-homo-Phenylalanin)-CONH—(CH-isobutyl)-B(OH)₂); the compound PS-352 (Phenyalanin-CH₂—CH₂—CONH—(CH-Phenylalanin)-CONH—(CH-isobutyl)l-B(OH)₂); the compound PS-383 (Pyridyl-CONH—(CHpF-Phenylalanin)-CONH—(CH-isobutyl)-B(OH)₂. The compounds already described in Adams et al. (1999) also count here. Alongside Epoxomicin and Eponemycin, peptidyl boric acid derivates have also shown themselves to be particularly suitable compounds. These proteasome inhibitors are very potent, very specific for the proteasome, block no other cellular proteases and so have as good as no side effects.

According to the invention, drugs were made available with the proteasome inhibitors which surprisingly

adversely affected the production of infectious offspring viruses by blocking the replication of influenza viruses, and thereby prevented the spread of an influenza virus infection in the organism;

block the release of infectious influenza viruses from infected cells;

limit the spread of an acute influenza virus infection;

suppress the viremia both in a new infection and also in returning RE infections with influenza viruses, and increase the success of virus elimination by the own immune system and/or by known drugs with a similar or different effect.

According to the invention, proteasome inhibitors find a use
in the treatment of influenza infections and related illnesses in humans and animals, which are caused by influenza viruses and related negative-strain RNA viruses,
as a drug for influencing, blocking or regulating the ubiquitin/proteasome pathway,
as a drug for influencing the enzymatic activities of the complete 26S proteasome complex and the free 20S catalytically active proteasome structure no assembled with regulatory subunits, and for selective blocking of the immunoproteasome.

The UPS inhibitors introduced according to the invention have a further use
for the prevention of an outbreak of illness and for the reduction of the infection outbreak in the organism of people already infected;
for prophylaxis of the establishment of a systemic influenza infection directly after contact with infectious biological samples, infected people or their near surroundings.

The UPS inhibitors introduced according to the invention can be administered both systemically and topically, preferably aerogenously. The active ingredient of a proteasome inhibitor introduced according to the invention can be introduced with at least one further antivirally-effective substance for prophylaxis and/or therapy of influenza infections.

The invention shall be described in greater detail on the basis of exemplary embodiments, without being limited to these examples.

### EXAMPLES

### Example 1

### Proteasome Inhibitor Shows No Toxic Side Effects after Aerosol Treatment of Mice

In order to investigate whether a proteasome inhibitor is toxic for mice after application as an aerosol, 6 mice were treated 3 times a day for 5 days with 500 nM of proteasome inhibitor. For this, 2 ml of proteasome inhibitor (500 nM) were nebulised with the help of a nebuliser (PARI®). The duration of treatment came to 10 minutes in each case. The treatment was carried out at 9:00, 12:00 and 15:00. As the test there were 6 mice which were treated with the solvent (DMSO/H₂O). In order to measure the body temperature and the body activity, mini-senders were implanted in the mice. The cages in which the mice found themselves stood on top of receiver plates. These receivers fed the signals to a computer which evaluated the data with the help of special software.

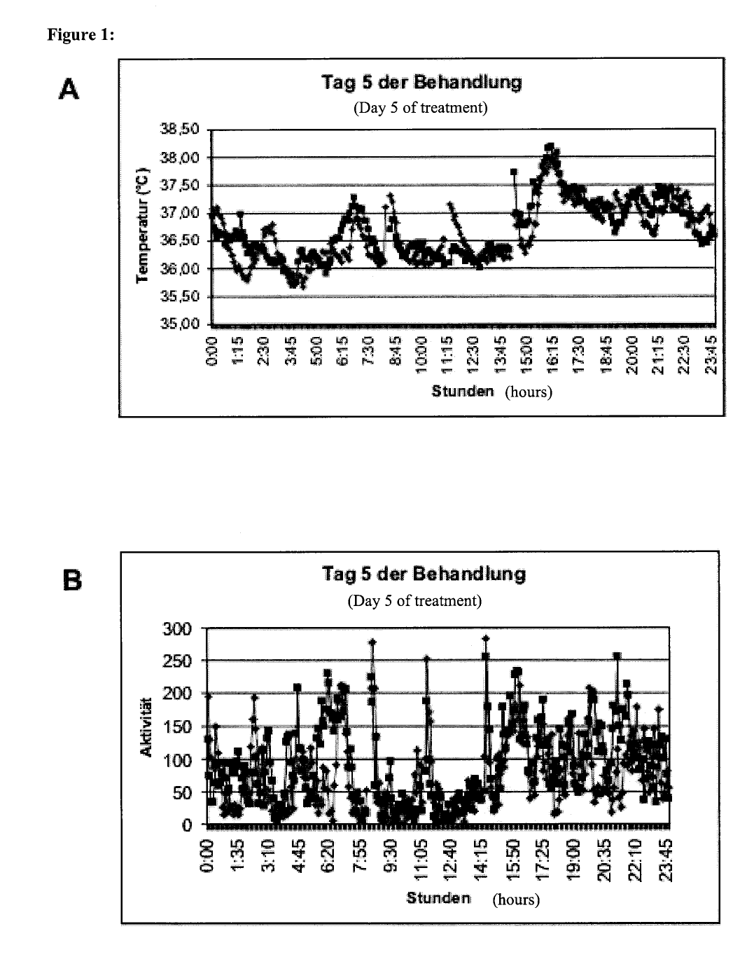

After implantation of the sender, the state of health of the mice was observed for 5 days, after which there took place the treatment with proteasome inhibitors for a further 5 days. During the treatment, no difference in body temperature (FIG. 1A) and body activity (FIG. 1B) could be established between mice which were treated with the proteasome inhibitor and the solvent. The graphics show the investigation values on the 5^{th} day of treatment as an example. The investigations show that mice, which were treated with the proteasome inhibitor in a concentration of 500 nM and a treatment period of 5 days showed no significant toxic effects. So the proteasome inhibitor in the named concentration is suitable for investigations for antiviral activity against influenza viruses in the mouse model.

### Example 2

### Proteasome Inhibitors Efficiently Block the Replication of Influenza Virus in a Concentration-Dependent Fashion and are not Significantly Toxic to the Host Cell in Antivirally Effective Concentrations in the Period of Observation

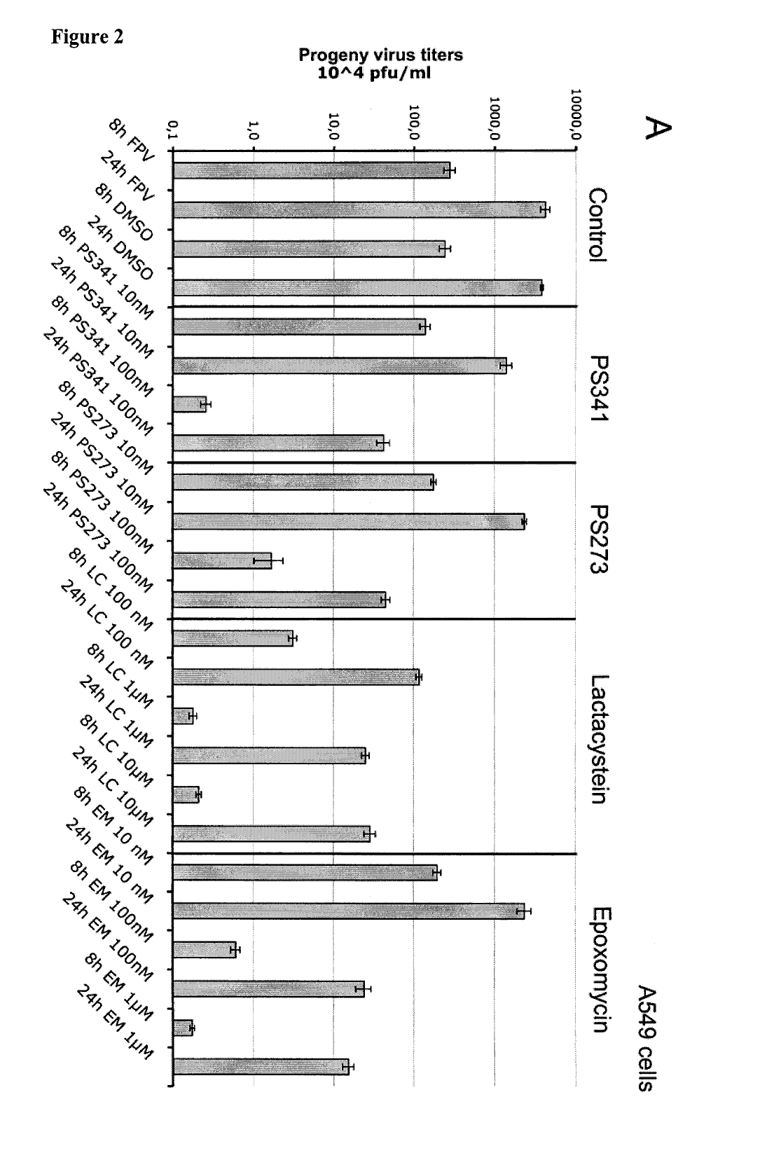

In order to investigate whether inhibitors of the proteasome have a negative effect on the replication of influenza viruses, human A549 lung epithelilum carcinoma cells (FIG. 2 A, B) or rather Madine Darby Canine Kidney (MDCKII) dog kidney epithelium carcinoma cells (FIG. 2 C) were pre-incubated with the proteasome inhibitors in the given concentrations for one hour, and then infected with the avian 'flu virus strain A/FPV/Bratislava/79 (H7N7) (multiplicity of the infection (MOI)=0.01). As a comparison there were untreated, infected cells, or rather cells treated with the solvent dimethylsulphoxide (DMSO). The substances introduced were PS341 (10 nM and 100 nM), PS273 (10 nM and 100 nM), lactacysteine (1 μM and 10 μM) as well as Epoxomycin (10 nM, 100 nM and 1 μM). At 8 and 24 h (FIG. 2A), or 8, 24 and 36 h (FIG. 2 B, C) after the beginning of the infection, the virus-containing medium supernatants were collected and the virus titre determined in plaque assays on MDCKIIcells.

The result showed that all inhibitors of the proteasome inhibit the replication of the aggressive influenza virus A/FPV/Bratislava/79 efficiently and in a concentration-dependent way.

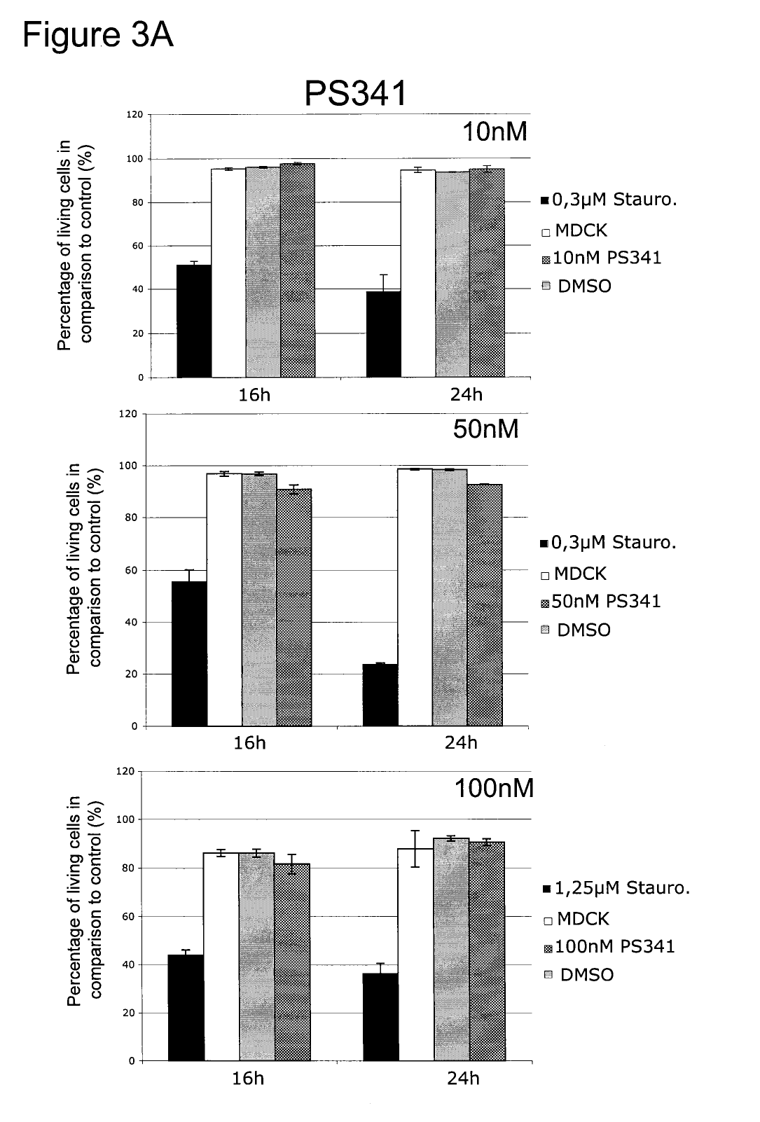

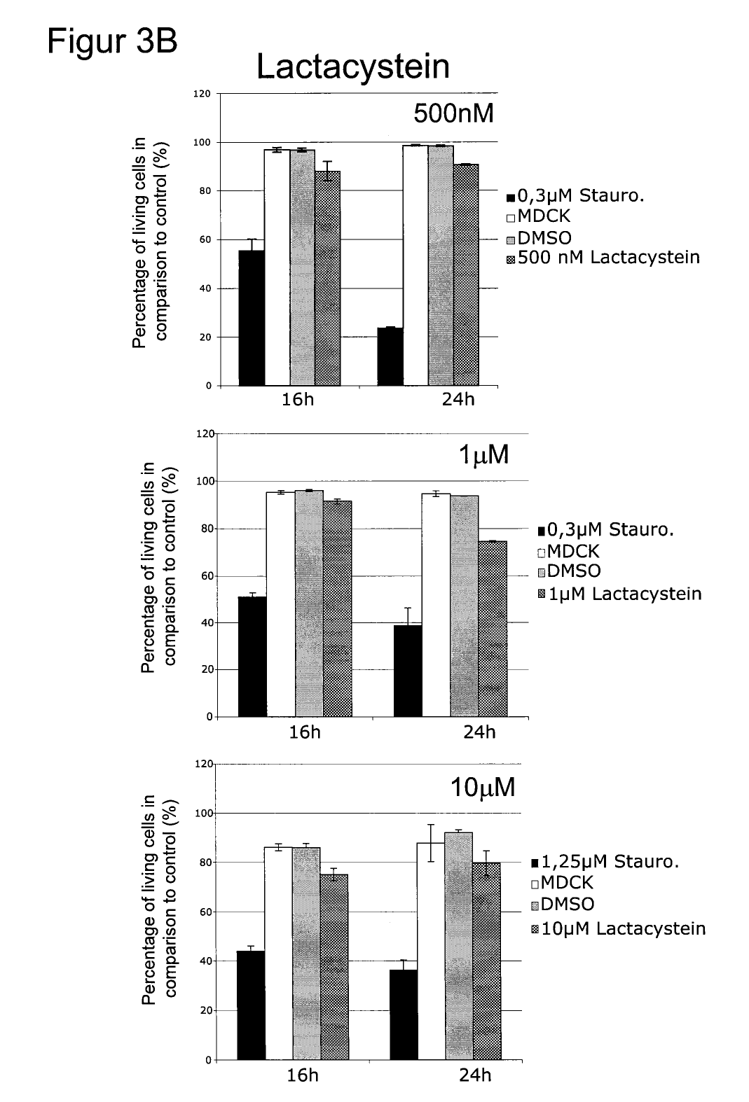

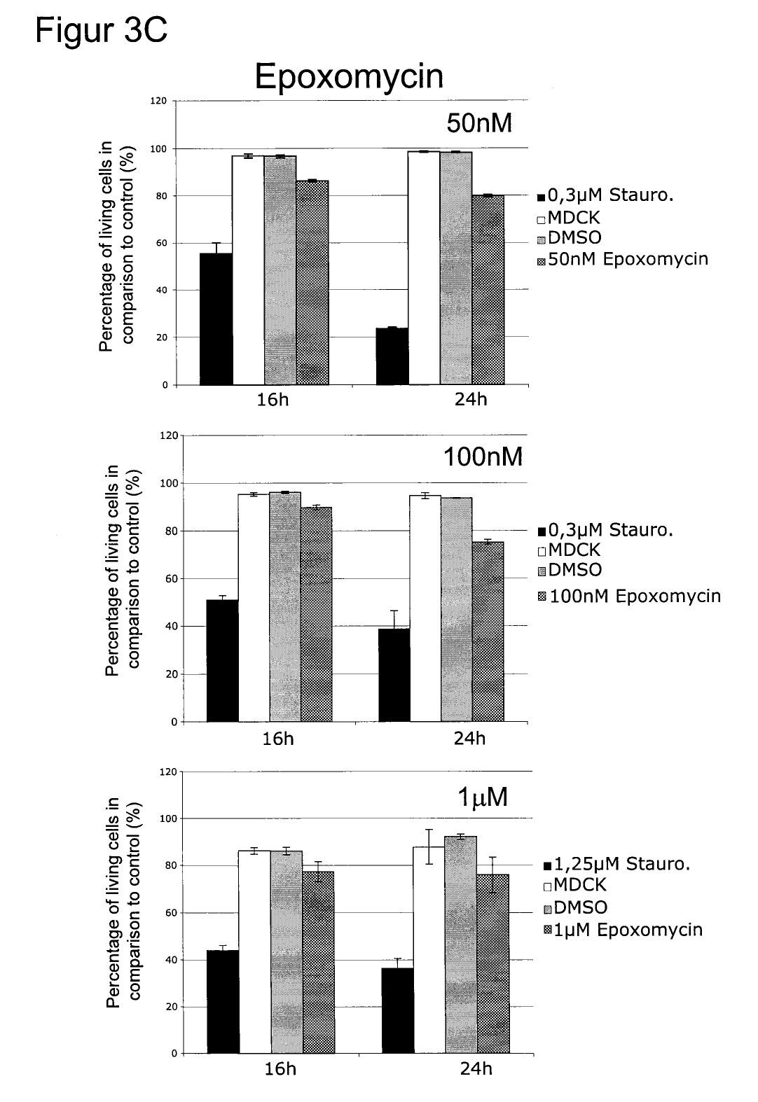

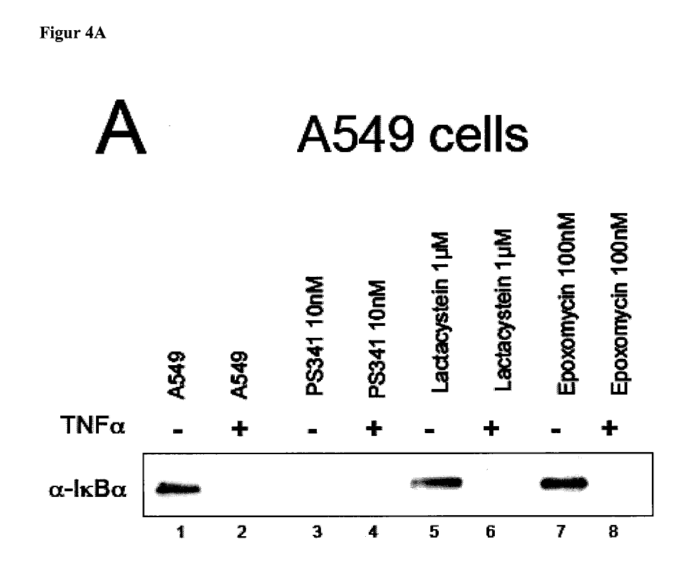

In order to answer the question as to whether the antiviral effect is set in action indirectly by a cellular toxic effect of the proteasome inhibitors, MDCKII cells (cell count: 2×10⁶) were treated with antivirally-effective concentrations of the most effective antiviral proteasome inhibitors PS341, Lactacysteine and Epoxomycin. As a control, the highly cytotoxic apoptosis-inducing substance Staurosporin (0.3 μM) was used. After 16 and 24 h, both the adherents and the dying already removed were collected, grown with PBS and treated with 50 μg/ml Propidiumiodid (PI). PI intercalates in the DNA strand dying cells. The analysis was carried out by means of throghflow cytometry (Becton Dickinson FACScan). In FIG. 3 (A, B and C), the percentage of dead cells in comparison to untreated control is depicted in each case.

It emerges that the proteasome inhibitors in antivirally-effective concentrations in the observation period of 24 h show no significant toxic effect. The idea that the antiviral effect of the proteasome inhibitors shown in FIG. 1 is based on toxic effects on the cells can therefore be ruled out.

### Example 3

### Proteasome Inhibitors have an Antiviral Effect Against Influenza Viruses in a Mechanism Independent of NF-κB

In order to investigate whether the antiviral effect of the proteasome inhibitors can be traced back to a blocking of the NF-κB-signal pathway, the TNFα-induced activation of NF-κB was analysed on the basis of the decomposition of the inhibiting protein IκBα (Inhibitor of κB) in the Western-Blot in the presence and absence of the proteasome inhibitors. For this, A549- (2×10⁶) or HEK293-cells (4×10⁶) were pre-incubated for 1 h with the proteasome inhibitors in various concentrations. Afterwards, the cells were treated for 15 min with 20 ng/nl of TNFα, in order to induce the decomposition of IκBα. The cells were subsequently grown with 1×PBS and lysed. The protein concentrations were determined by means of Bradford Protein Assay (Biorad) and brought into line with one another. The proteins were separated by means of SDS gel electrophoresis and transferred to a nitrocellulose membrane. The IκBα-decomposition was made visible by means of a IκBα specific antiserum (Santa Cruz Biotechnologies) and a horseradish perodidase-coupled secondary reagent (Amersham) with the help of an electrochemical luminescence reaction (ECL, Amersham).

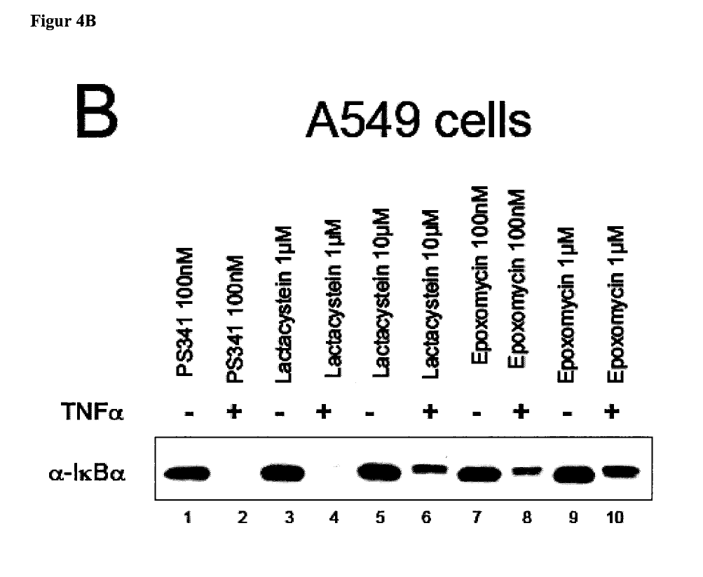

Surprisingly, antivirally-effective concentrations of each of the proteasome inhibitors were not in a position to effectively inhibit the TNFα-induced decomposition of IκBα and thereby to block the NF-κB activation. So, for example, PS341 (100 nM) was not in a position to prevent the IκBα-degradation in A549- or HEK293-cells (FIGS. 4B and D). Likewise, the same concentration of PS341 in infected A549 cells led to a reduction of the virus titre (over 2 logarithmic levels after 8 h, see FIG. 2A). Similarly, lactacysteine (1 μM) led to a reduction of the virus titre (FIG. 2A), but was not effective enough to block IκBα-degradation (FIGS. 4B and D).

This unequivocally proves that the proteasome inhibitors act antivirally by another mechanism than the blocking of NF-κB.

### Example 4

### A Pharmacological Inhibitor of the Proteasome MG132 Interferes with the Influenza Virus Induced Expression of Proapoptitic Genes and Efficiently Inhibits Influenza Virus Replication In Vitro and In Vivo

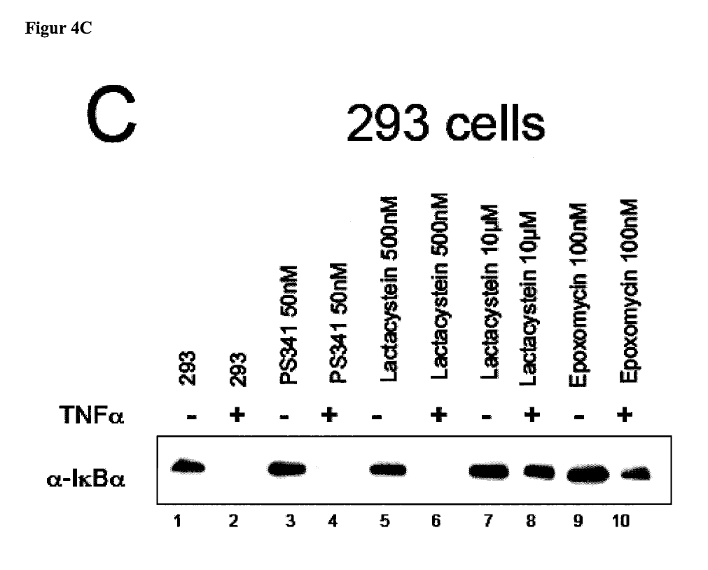

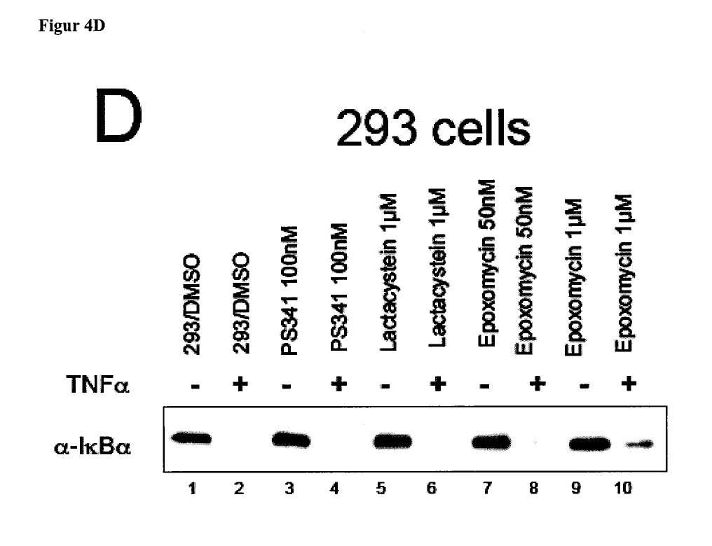

In order to investigate whether the proteasome inhibitor MG132 has a negative effect on the replication of influenza viruses, the human lung epithelium cell line A549 was infected with the highly pathogenic avian influenza A virus A/FPV/Bratislava/79 (multiplicity of the infection=1) in the presence or absence of various concentrations of the proteasome inhibitors MG132 (1, 5, 10 μM). In the case of presence of the inhibitor, the cells were pre-incubated with the substance 30 minutes before infection. 24 h after infection, the cell supernatants were investigated for the content of infectious offspring viruses in plaque assays. As a result, in the presence of MG132, a concentration-dependent reduction of up to 10 times of the virus titre showed itself (FIG. 5).

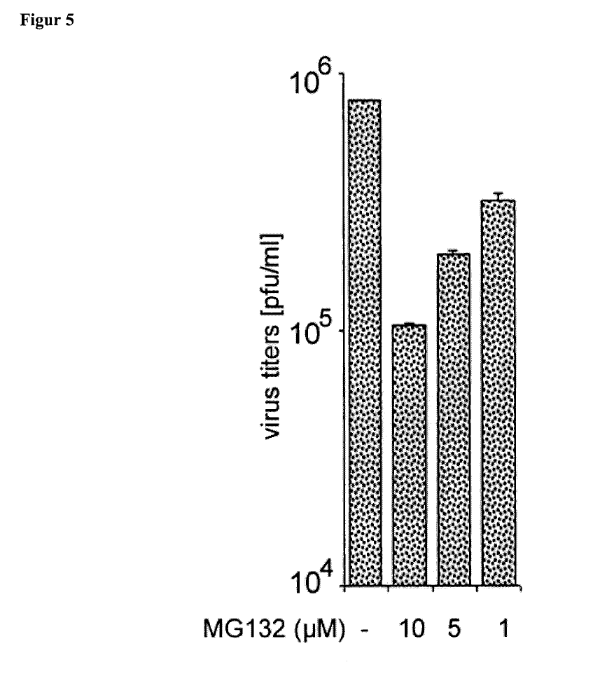

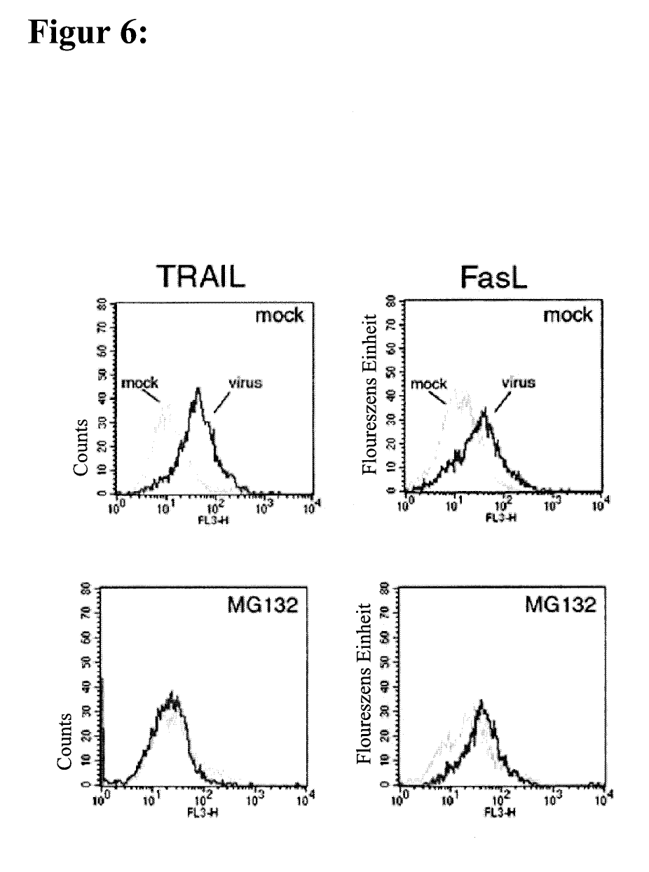

In order to investigate whether the reduction of the virus titre is accompanied by a reduced expression of the proapoptotic ligands TRAIL and FasL/CD95L, the A549 lung epithelium cell line was infected as described with the avian influenza virus A/FPV/Bratislava/79 (multiplicity of the virus=1) in the presence or absence of the proteasome inhibitor MG132 (10 μM). Around 24 h after infection, the cells were fixed with 4% paraformaldehyde and incubated with specific antibodies against TRAIL and FasL/CD95L. After connection of the antibodies with a fluorescent dye, the cells were subjected to a throughflow cytometric analysis (FACS) in order to measure the expression of the proapoptotic factors. As a result, the FACS profiles show a clearly reduced virus-induced expression of FasL and TRAIL in the presence of MG132 (FIG. 6).

In order to test the antiviral activity of MG132 in an in vivo infection model in the mouse, C57B1/6 mice (10 weeks old, own breed of BFAV Tübingen) were intranasally infected with 104 infectious units of the mouse-adapted, highly pathogenic avian influenza A virus A/FPV/Bratislava/79, and left untreated (n=14), or rather treated in an isolation cage 5 times daily with a nebulised aerosol of 1 mM MG132 (n=8) for 5 days. The treatment took place once a day, beginning 1 h before the infection on day 1. The survival rate of the mice was determined.

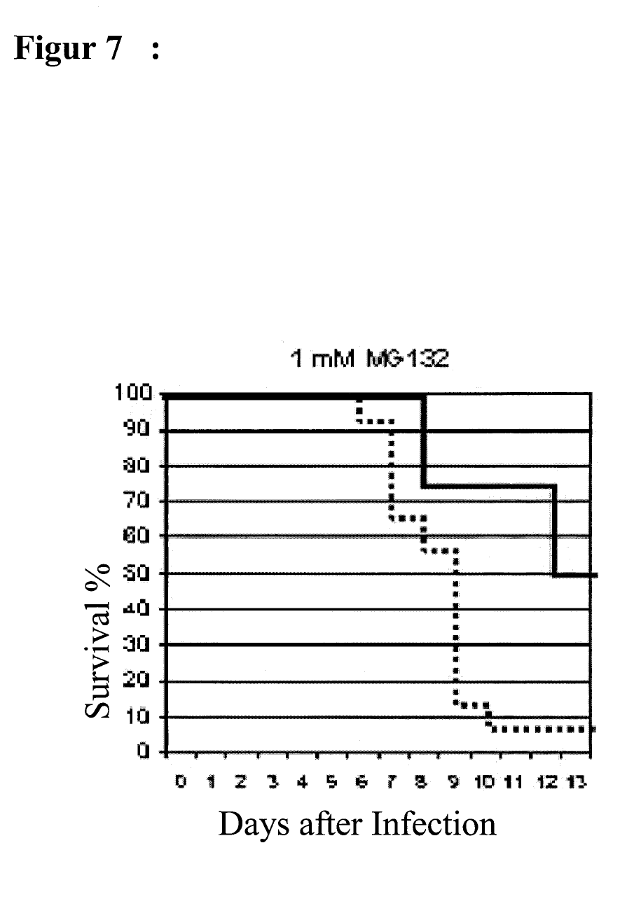

As a result, overall a significantly increased survival rate of the infected and MG132-treated mice was shown in comparison to the untreated controls (FIG. 7).

### Example 5

### Material and Methods

Treatment of mice with proteasome inhibitors: The treatment of mice was carried out in an inhalation system. For this, 6 mice were treated in inhalation tubes. These 6 tubes stood in connection with a central cylinder with a total volume of 8.1×10⁻⁴ m³. A PARI® nebuliser (Aerosol Nebuliser; Art. no. 73-1963) was connected to the central cylinder. Proteasome inhibitors or solvents were nebulised with 1.5 bar for 10 min (approx. 2 ml) in the chamber. BALB/c mice were treated 3 times a day at 9:00, 12:00 and 3:00 for 5 days. The general state of health of the mice was checked twice a day and the animals was weighed once a day.

Mouse monitoring: The monitoring of the body temperature and the body activity of the mice was carried out with the Vital View® Software and Hardware System (Mini Mitter U.S.A.). This system allows for the generation of physiological parameters of the mouse. The hardware is composed of a transmitter (E-mitter)/receiver system. The E-mitter collects data of the body temperature and the body activity of the animals. These data are registered every 5 minutes and forwarded onto a PC. There the analysis of the data is carried out with the help of the Vital View Software.

For the implantation of the E-mitter, the mice are anaesthetised by intraperentonal injection of 150 μl Ketamin Rompun. The mice's stomach is shaved and an approx. 1.5 cm-large cut is made along the Linea abla to open up the abdomen. Afterwards, the E-mitter is positioned and the opening is closed with wound clamps (autoclip 9 mm; Becton & Dickinson, Germany). The animals are brought back into their cages and the successful implantation is checked with the help of the Vital View Software.

Viral infection of cells: Cells are grown and the virus solution diluted in infection PBS (PBS with 1% penicillin/streptomycin, 1% Ca²⁺/Mg²⁺, 0.6% bovine albumin 35%) is added. The cells were incubated with the virus in the given quantities for 30 min at 37° C. in the incubator and subsequently grown anew, in order to remove the virus particles which had not bonded to cells.

After this adsorption phase, the cells were covered with infection medium (MEM with 1+% penicillin/streptomyci and 1% Ca²⁺/Mg²⁺). The cells were kept in the incubator at 37° C. until cell harvest or determination of the remaining offspring viruses.

Plaque-Assay for determining infectious Offspring viruses: In order to determine the number of infectious particles in a virus solution, plaque-assays were carried out on MDCK II cells. Infection of the cells was carried out with a row of virus solution diluted per 500 μl logarithmically in infection PBS. Incubation was carried out in the incubator for 30 min at 37° C., after which the virus solution was removed and the cells were covered with a mixture of medium and agar (27 ml ddH₂O, 5 ml 10×MEM, 0.5 ml penicillin/streptomycin, 1.4 ml sodium bicarbonate, 0.5 ml 1% DEAE-dextrane, 0.3 ml bovine albumin, 15 ml 3% oxoid agar; 500 μl Ca²⁺/Mg²⁺). The cells were kept at room temperature until setting of the medium/agar mixture and afterwards incubated for 2 to 3 days at 37° C. in the incubator until there was a build-up of visible plaques of lysed cells.

The plaques were dyed with 1 ml of a neutral red PBD solution for a further 1-2 hours in the incubator until the plaques became visible.

Cell dyeing with propidium iodide: The substance propidium iodide can penetrate through the cell membrane of dying cells and intercalated into the DNA in the cell nucleus. The number of dying and dead cells can be determined according to their fluorescence in the throughflow cytometer. MDCK cells (2×10⁶) were treated with the given concentrations of proteaome inhibitors. MDCK cells were treated with the apoptosis-releasing substance Staurosporin (0.3 μM) as a toxicity test. After 16 or 24 h, the adherent cells as well as cells left over were collected and dyed with 50 μg/ml propidium iodide. The analysis was carried out with the help of throughflow cytometry (BD FACScan).

SDS Gelelectrophoresis and Western-Blot: The cells to be lysed were first grown with PBS and subsequently provided per Well with a 6-Well plate with 200 μl of the lysis buffer RIPA (25 mM tris pH 8, 137 mM NaCl, 10% glycerine, 0.1% SDS, 0.5% sodium deoxycholate DOC, 1% NP40, 2 mM EDTA pH 8, freshly added: Pefablock 1:1000, Aprotinin 1:1000, Leupeptin 1:1000, sodium vanadate 1:100, benzamidine 1:200). The cells were lysed with swinging at 4° C. for 30 min, and subsequently at 14 000 rotations per minute for 10 min at 4° C. in the table centrifuge, in order to separate the proteins from the cell ruins.

The protein concentration in the lysates was determined with the help of the Biorad protein dye solution (Biorad) and set at the same protein volumes. Afterwards, the samples were replaced with a 5× sample buffer (10% SDS, 50% glycerine, 25% β-mercaptoethanol, 0.01% bromophenol blue, 312 mM tris). The β-mercaptoethanol in the sample buffer additionally takes care of the protein denaturation by reducing the disulphide bonds. The negatively-charged proteins therefore move through the electric field towards the positive electrode, wherein larger proteins in the gel are held back more strongly. The electrophoresis gel consists of a 5% combining gel (0.49 ml rotiphoresis gel 30, 3.25 ml stacking buffer (0.14 M tris pH 6.8, 0.11% temed, 0.11% SDS), 45 μl 10% ammonium persulphate), in which the proteins are concentrated, and of a 10% dividing gel (3.375 ml rotiphoresis gel 30, 2.5 ml running buffer (1.5 M tris pH 9, 0.4% temed, 0.4% SDS), 4.025 ml aqua bidest, 200 μl 10% ammonium peroxodisulphate), in which the proteins are separated according to their molecular weight.

The gel is poured into two glass plates kept in a pouring state and held at a distance by a spacer, wherein the separating gel is poured first. This was covered with isopropanol for depolymerisation and subsequently grown with acqua bidest. The combining gel is poured onto the dividing gel and placed bubble-free in a sample chamber. After depolymerisation the sample chamber is drawn and the gel placed in an electrophoresis chamber which was filled with 1×SDS-PAGE-Puffer (5 mM tris, 50 mM glycerine, 0.02% SDS). The denatured proteins and a marker are filled into the bags. The gel runs with a constant flow of 25-40 mA.

Afterwards, the proteins are transferred by means of an electrical field from the gel onto a nitrocellulose membrane. Proteins which are immobilised on the nitrocellulose membrane can be detected by a specific antibody. This is identified by an enzyme-connected, second antibody, upon which the protein can be made visible by a chemoluminscent reaction. In so doing, the lumino substrate is oxidised by a Horseradish peroxidase bonded to the secondary antibody, through which it moves into an excited state and emits light, which can be made visible on an X-ray film.

The SDS gel with the dividing proteins is taken up by the pouring apparatus and laid on two Whatman papers soaked with blotting buffer (3.9 mM glycine, 4.8 mM tris, 0.0037% SDS, 10% methanol). The nitrocellulose membrane was laid bubble-free onto the gel before the exciting takes place in a wet blot chamber (BioRad) filled with blotting buffer. The proteins are transferred under a constant electrical current of 400 nA onto the nitrocellulose membrane in 50 min. In this the proteins move from the cathode to the anode.

According to the blotting procedure, non-specific binding sites are blocked with 5% milk powder in 1×TBST (50 μM tris, 0.9% NaCl, 0.05% Tween 20, pH 7.6) for at least 45 min being swung at room temperature, in order to prevent a non-specific binding of the antibody to the membrane. The membrane is subsequently incubated with the primary antibodies (here—anti IκBα, dilution 1:1000, Santa Cruz Biotechnologies) overnight at 4° C. while rocking. The membrane is washed three times, for 10 min each time with 1×TBST, in order not to remove non-bonded residues of the antibody. Afterwards, the membrane is rinsed for 1-2 hours in the secondary antibody at room temperature.

After another three washes with 1×TBST, the membrane is brought into the condition of an increased chemoluminescence (ECL=enhanced chemoluminescence) by the addition of a chemoluminescence substrate (250 mM luminol, 90 mM p-coumaric acid, 1 M tris/HCl pH 8.5, 35% H₂O₂), a condition in which the luminol substrate contained in it is replaced by the Horseradish peroxidase bonded to the secondary antibody. The membrane is incubated for 1 min with the substrate, then dried and then laid into an X-ray film cassette, in which the increased chemoluminescence is made visible on an X-ray film.

Throughflow cytometric analysis: TRAIL and FasL Expression were proven with the help of an intracellular fluorescent dye with connected antibodies. A549 cells were infected with the virus strain A/FPV/Bratislava/79 (H7N7) (MOI=5) for 8 hours in the presence of 2 μM monensin in order to prevent protein secretion. As a result, the cells were fixed with 4% paraformaldehyde at 4° C. for 20 min and then washed with permeability buffer (0.1% saponin/1% FBS/PBS). Then there took place incubation with the primary antibodies against TRAIL, FasL or an isotope test (antibodies from Becton Dickinson). Next the cells were dyed with Biotin-Sp-conjugated goat anti-mouse IgG (Dianova) and streptavidine-Cychrome (Becton Dickinson). The fluorescence was measured in the FL3 channel of a FACScalibur throughflow cytometer (Becton Dickinson).

Infection and treatment of mice: 10 week-old C57B1/6 mice (own breed, FLI, Tübingen) were introduced for infection and treatment. The treatment with approx. 2 ml of a 1 mM MG132 (Sigma) dilution was carried out once a day by means of aerosol administration in a cage inhalation, beginning one hour before intranasal infection with 5×10³ to 10⁴ plaque-forming infectious units (pfu) of the virus strain A/FPV/Bratislava/79 (H7N7). Nebulisation of the MG132 solution was carried out by means of a mouse Minivent System (Hugo Sachs Electronics—Harvard Apparatus) combined with a Nebuliser (Hugo Sachs Electronics—Harvard Apparatus).

### LIST OF ABBREVIATIONS

DNA desoxyribonucleic acid
kDa Kilodalton (measure of molecular weight)
Ki inhibitory constants

### LC Lactacystine

### MDa Mega Dalton

### MHC Major Histocompatibility Complex

NLVS Proteasom-Inhibitor z-Leuzinyl-Leuzinyl-Leuzinyl-vinylsulfon (NLVS)
PGPH Postglutamyl-Peptide hydrolysing
PI Proteasome inhibitor
PCR polymerase chain reaction
RNA Ribonucleic acid

### RSV Rous Sarcoma Virus

RT reverse transcriptase

### Ub Ubiquitin

UPS Ubiquitin/Proteasome system
Vero-Zellen human permanent transformed cells of the VERO line
Vpr HIV-1 protein Vpr
zLLL Tripeptidaldehyde N-carbobenzoxyl-L-leucinyl-L-leucinyl-L-leucinal

### BIBLIOGRAPHY

Adams, J., Ma, Y., Stein, R., Baevsky, M., Greiner, L., and Plamondon, L. (1996) Boronic ester and acid compounds, synthesis and uses. U.S. Pat. No. 1,448,012TW01.
Adams, J., Palombella, V. J., Sausville, E. A., Johnson, J., Destree, A., Lazarus, D. D., Maas, J. Pien, C. S., Prakash, S., Elliott, P. J. (1999) Proteasome inhibitors: a novel class of potent and effective antitumor agents. Cancer Res. 59:2615-2622.
Adams, J., Palombella, V. J., Elliot, P. J. (2000) Proteasome inhibition: a new strategy in cancer treatment. Investigational New drugs 18:109-121.
Chen et al., Nat. Med. 7, 1306-1312, 2001
Ciechanover, A., Orian, A., Schwartz, A. L. (2000) The ubiquitin-mediated proteolytic pathway: Mode of action and clinical implications. J. Cell. Biochem. 77:40-51.
Drosten C, Gunther S, Preiser W, Van Der Werf S, Brodt H R, Becker S, Rabenau H, Panning M, Kolesnikova L, Fouchier R A, Berger A, Burguiere A M, Cinatl J, Eickmann M, Escriou N, Grywna K, Kramme S, Manuguerra J C, Muller S, Rickerts V, Sturmer M, Vieth S, Klenk H D, Osterhaus A D, Schmitz H, Doerr H W. Identification of a
Elliott, PJ, Ross J S; (2001). The proteasome: a new target for novel drug therapies. Am J Clin Pathol. 116: 637-46.
Fenteany, G; Standaert, RF; Lane, WS; Choi, S; Corey, EJ; Schreiber, SL (1995). Inhibition of proteasome activities and subunit-specific amino-terminal threonine modification by lacta-cystein. Science 268: 726-731.
Gubareva et al J Infect Dis 178, 1257-1262, 1998.
Hershko, A., Ciechanover, A. (1998) The ubiquitin system. Annu Rev Biochem. 67:425-479.
Harty, RN; Brown, M E; Wang, G; Huibregtse, J; Hayes, FP; (2000). A PP×Y motif within the VP40 protein of Ebola virus interacts physically and functionally with a ubiquitin ligase: implications for filovirus budding. Proc. Natl. Acad. Sci. USA 97: 13871-13876.
Lamb and Krug, in Fields, Virology, Philadelphia: Lippincott-Raven Publishers, 1353-1395, 1996.
Lee N, Hui D, Wu A, Chan P, Cameron P, Joynt G M, Ahuja A, Yung M Y, Leung C B, To K F, Lui S F, Szeto C C, Chung S, Sung J J. A Major Outbreak of Severe Acute Respiratory Syndrome in Hong Kong. N Engl J Med 2003 Apr. 14; [epub ahead of print].
Ludwig et al., Trends Mol. Med. 9, 46-51, 2003.
Ludwig et al., FEBS Lett 561, 37-43, 2004.
Meng, L; Kwok, BH; Sin, N; Crews, CM (1999a). Eponemycin exerts its antitumor effect through the inhibition of proteasome function. Cancer Res. 59:2798-2801.
Qui et al. Nature 437, 1108, 2005.
Patnaik, A, Chau, V, Wills, JW; (2000). Ubiquitin is part of the retrovirus budding machinery. Proc. Natl. Acad. Sci. USA 97:13069-13074.
Poutanen S M, Low D E, Henry B, Finkelstein S, Rose D, Green K, Tellier R, Draker R, Adachi D, Ayers M, Chan A K, Skowronski D M, Salit I, Simor A E, Slutsky A S, Doyle P W, Krajden M, Petric M, Brunham R C, McGeer AJ. Identification of Severe Acute Respiratory Syndrome in Canada. N Engl J Med 2003 Apr. 10.
Rock, K. L., Goldberg, A. L. (1999) Degradation of cell proteins and the generation of MHC class I-presented peptides. Annu. Rev. Immunol. 17:739-779.
Scholtissek and Muller, Arch Virol 119, 111-118, 1991.
Schubert, U., Ott, D. E., Chertova, E. N., Welker, R., Tessmer, U., Princiotta, M. F., Bennink, J. R., Krausslich, H.-G., Yewdell, J. W. (2000a) Proteasome inhibition interferes with gag polyprotein processing, release, and maturation of HIV-1 and HIV-2. Proc. Natl. Acad. Sci. USA 97:13057-13062.
Strack, B; Calistri, A; Accola, M A; Palu, G; Göttlinger, HG (2000). A role for ubiquitin ligase recruitment in retrovirus release. Proc. Natl. Acad. Sci. USA 97:13063-13068.
Webster et al., Microbiol Rev, 56, 152-79, 1992

1. A method for treating an orthomyxovirus infection in a subject in need of such treatment, the method comprising administering an effective amount of at least one proteasome inhibitor and/or at least one inhibitor of the ubiquitin proteasome pathway (UPS).

2. The method according to claim 1, wherein the at least one inhibitor selected from the group consisting of a proteasome inhibitor, a ubiquitin ligase inhibitor, and a ubiquitin hydrolases inhibitor.

3. The method according to claim 1, wherein the at least one inhibitor has specificity for a 26S proteasome of a host cell.

4. The method according to claim 3, wherein the at least one inhibitor interacts only with a catalytically active hydroxyl-threonin group of a beta subunit of the 26S proteasome, and specifically blocks only the proteasome.

5. The method according to claim 3, where when the at least one inhibitor is accepted by a cell in the subject, the at least one inhibitor selectively blocks an individual enzymatic activity of the 26S proteasome and selectively blocks specific assembly of the proteasome.

6. The method according to claim 5, wherein the at least one inhibitor blocks specific assembly of an immunoproteasome.

7. The method according to claim 1, wherein the at least one inhibitor is A ubiquitin-conjugating enzyme inhibitor, a ubiquitin-hydrolysing enzyme inhibitor, or both.

8. The method according to claim 1, wherein the at least one inhibitor
induces apoptosis in an influenza-infected cell in the subject; and
disrupts release and production of infectious virus particles by inhibiting assembly and maturation of the orthomyxovirus.

9. The method according to claim 1, wherein the orthomyxovirus is an influenza virus.

10. The method according to claim 1, which further comprises administering one or more antiviral compositions.

11. The method according to claim 10, wherein the one or more antiviral compositions is selected from the group consisting of ripavarin, an interleukin, a nucleoside analogue, a protease inhibitor, a viral kinase inhibitor, a membrane fusion inhibitor, virus entry inhibitor.

12. The method according to claim 10, wherein the one or more antiviral compositions is a neuraminidase inhibitor, or an M2 ion channel IAV protein inhibitor.

13. The method according to claim 1, wherein treating the infection comprises treating illness in the subject.

14. The method according to claim 1, wherein treating comprise reducing spread of infection.

15. The method according to claim 1, wherein treating comprises treating a grippal infection.

16. The method according to claim 1, which further comprises administering at least one drug targeting a ubiquitin-conjugating enzyme, a ubiquitin-hydrolising enzyme, a cellular factor which interacts with ubiquitin.

17. The method according to claim 1, which comprises administering peptide derivates comprising a C-terminal epoxyketone structure, a β-lactone derivative, aclacinomycin A, lactacystine, chemically-modified lactacystine variants, N-carbobenzoxy-L-leucinyl-L-leucinyl-L-leucinal, MG232, N-carbobenzoxy-Leu-Leu-Nva-H, N-acetyl-L-leucinyl-L-leucinyl-L-norleucinal, N-carbobenzoxy-ile-glu(OBut)-ala-carbobenzoxy-ile-Glu(OBut)-ala-leu-H, a peptide with a C-terminal α,β-epoxyketone structure, a peptide with a C-terminalvinyl sulphone, a peptide with a C-terminal glyoxal residue, a peptide with a C-terminal boric acid residue, or a peptide with a C-terminal pinacol-ester.

18. The method according to claim 17, which comprises administering a C-terminal vinyl sulphone, which is carbobenzoxy-L-leucinyl-L-leucinyl-L-leucin-vinyl-sulphone or 4-hydroxy-5-iodo-3-nitrophenylactetyl-L-leucinyl-L-leucinyl-L-leucin-vinyl-sulphone (NLVS)

19. The method according to claim 17, which comprises administering a peptide with a C-terminal glyoxal or a peptide with a C-terminal boric acid residue, which is pyrazyl-CONH(CHPhe)CONH(CHisobutyl)B(OH)2) or dipeptidyl-boric acid derivates

20. The method according to claim 17, which comprises administering a peptide with a C-terminal pinacol-ester, which is benzyloxycarbonyl(Cbz)-leu-leu-boroLeu-Pinacol-Ester.

21. The method according to claim 17, which comprises administering a peptide with a C-terminal epoxyketone structure selected from the group consisting of epoxomicin and eponemycin.

22. The method according to claim 1, which comprise administering at least one inhibitor selected from the group consisting of PS-519, 1R-[1S,4R,5S]]-1-(1-Hydroxy-2-methylpropyl)-4-propyl-6-oxa-2-azabicyclo[3.2.0]heptane-3,7-dione, PS-303 (NH₂(CH-naphthyl)-CONH—(CH-isobutyl)-B(OH)₂), PS-321 (morpholine-CONH—(CH-napthyl)-CONH—(CH-phenylalanine)-B(OH)₂), PS-334 (CH₃—NH—(CH-naphthyl-CONH—(CH-isobutyl)-B(OH)₂), PS-325 (2-Quinol-CONH—(CH-homo-phenylalanine)-CONH—(CH-isobutyl)-B(OH)₂), PS-352 (phenyalanine-CH₂—CH₂—CONH—(CH-phenylalanine)-CONH—(CH-isobutyl)l-B(OH)₂), PS-383 (pyridyl-CONH—(CHpF-phenylalanine)-CONH—(CH-isobutyl)-B(OH)₂).

23. The method according to claim 1, wherein the at least one inhibitor is administered systemically.

24. The method according to claim 1, wherein the at least one inhibitor is administered topically.

25. The method according to claim 1, wherein the at least one inhibitor is aerogenously administered.
